(19) United States
(12) Patent Application Publication
(10) Pub. No.: US 2019/0244155 A1
(43) Pub. Date: Aug. 8, 2019

(54) TASK MANAGEMENT METHOD

(71) Applicant: SALAMANDER U, Ohey (BE)

(72) Inventors: Denis DECUBBER, Flobecq (BE); Claude DEDRY, Archennes (BE); Raphaël BENEDET, Liège (BE); Stéphane FOCANT, Watermael-Boitsfort (BE); Jean-Sébastier DECUBBER, Flobecq (BE); Manuel BASILAVECCHIA, Sombreffe (Tongrinne) (BE)

(21) Appl. No.: 16/340,159

(22) PCT Filed: Oct. 10, 2017
(86) PCT No.: PCT/EP2017/075793

§ 371 Date: Apr. 8, 2019

(30) Foreign Application Priority Data
Oct. 25, 2016 (BE) 2016/5805

(51) Int. Cl. G06Q 10/06; G10L 15/22; G16H 20/10

(52) U.S. Cl. CPC G06Q 10/063114; G10L 2015/223; G16H 20/10; G10L 15/22

(57) ABSTRACT

The present invention proposes a method for managing tasks of a given recipe to be carried out by at least one operator in a regulatory environment, whereby
the operator receives from a computer system voice commands to execute the tasks of the recipe,
the operator executes the tasks,
the operator verbally reports to the computer system on the task execution and
The task execution is verified by the computer system.

The invention concerns the field of industrial production wherein precise, regulated and controlled procedures must be followed by operators.

Although industrial production is increasingly automated, some types of production may only be carried out by operators. This is the case, for example, for textile productions or certain agri-food productions.

This is also the case for biological or pharmaceutical productions, such as vaccines or cellular or gene therapies, which must be done in a sterile environment, according to very strict standards and must be subject to constant quality control. The procedures, or recipes, established for these productions must be strictly followed by operators, and a certain number of parameters must be measured and recorded during the production cycle.

Usually, for these sterile clean room productions, a procedure, listing the tasks to be carried out and the framework to be respected for the parameters related to these tasks, is drafted by the production managers. It is then printed on paper, decontaminated by irradiation, transmitted to a team of operators who take the printed procedure with them into a clean room where they must wear a full set of protective gear—suit, gloves, mask, glasses, etc. One of the operators is in charge of manual operations, the second operator is in charge in particular of reading the procedure to his colleague and noting the parameters measured by the same. In effect, the operator performing the manual operations is equipped with sterile gloves or has his arms inserted in a glovebox or laminar flow and cannot, at the same time, handle a form and a pencil. This operator may also need glasses to read written documents, while not needing them to perform the task. If the operator were alone, he would have to constantly take off and put on his glasses and/or some equipment to read the procedure.

At the end of the production cycle, the notes taken by the operator are transmitted to the personnel in charge of verifying and validating the quality of the production before being able to authorize the release of the product batch and placing it on the market.

It is also possible that instead of being printed, the procedure may be available to read on a computer screen. In this case, the second operator reads the instructions to his colleague from the computer screen and records the measured parameters in the computer.

Whether the procedure is read on paper or on a computer screen, the second operator is essential, both to free the hands of the operator performing the operations and to double-check the operations performed by his colleague.

These production characteristics are not only found in sterile clean rooms. Some of these characteristics are also applied in the agri-food sector, where the cold chain must be scrupulously respected, or in the microelectronics sector, where production is carried out in dust-free clean rooms.

This production method has a number of drawbacks. First, the use of a printed procedure requires a preliminary step of decontamination of the paper when it is introduced into the sterile or dust-free room. It is then necessary to retranscribe the measured parameters on the documents.

Secondly, having two operators carry out the production instead of one has a significant impact on the production cost. This cost is particularly prohibitive for small productions, such as, for example, cellular or gene therapies, where the batch produced may be different for each patient (as in the case of autologous productions).

The production cycle must also be completed before being subjected to quality control. A batch may thus be rejected after it has been fully completed, whereas the problem leading to its rejection occurred at the beginning of the production stages. Unnecessary costs are then implemented, both in terms of time spent by operators and in terms of material resources that could have been saved.

Finally, the margin of error due to the subjectivity of operators in their decision-making during their tasks is considerable. This leads to variability in the quality of batches produced, particularly between batches produced by different operators.

It was thus considered necessary by the applicant to propose a production method and a tool for the implementation of this method allowing the aforesaid problems to be eliminated.

To this end, the present invention proposes, first, a method for managing tasks of a given recipe to be carried out by at least one operator in a regulatory environment, whereby
the operator receives from a computer system voice commands to execute the tasks of the recipe,
the operator executes the tasks,
the operator reports verbally to the computer system on the task execution and
the task execution is verified through the computer system, the verification of task execution comprising the comparison of the information reported by the operator with the expected information previously entered in the recipe, said expected information being one of the elements of the group comprising a value, a range of values, a term and an expression and the verification integrating the result of the verification of the tasks in the selection of a new order to be issued.

Due to the invention, a single operator may carry out the tasks of a procedure to be followed, with quality control and decision-making support for each task, in order to reduce production costs while improving quality.

Advantageously, for the execution of a task, a dialogue may be established between the operator and the computer system.

The recipe may be entered into the computer system beforehand.

The computer system may also interface with other computer systems to monitor the task execution.

The task execution may be stored in the computer system. The computer system may then generate a report on the task execution or participate in the generation of this report.

A regulatory environment means an environment subject to a set of rules. These rules may be based on specific legislation relating to compliance with health, safety or quality standards. They may also be based on internal regulations within a company or organization and relating to specifications to be respected or constraints defined by the company or organization.

The present invention also proposes, as an intermediate product, an algorithm for managing tasks of a given recipe to be carried out by at least one operator in a regulatory environment for the implementation of the method of the invention, designed to
issue voice commands to the operator to execute the tasks of the recipe,
receive a verbal report on the task execution and
participate in verifying the task execution.

Advantageously, the algorithm is designed to integrate the result of verifying the task into the selection of a new order to be issued.

The algorithm may also comprise a module to help in drafting the recipe.

In order to improve the quality of interaction between the computer system and the operator, the algorithm comprises a speech recognition module associated with a specific grammar to receive the verbal report.

The algorithm may possibly be designed to integrate external data to verify the task execution.

The invention will be better understood by means of the following description of several embodiments of the invention, with reference to the accompanying drawing wherein:

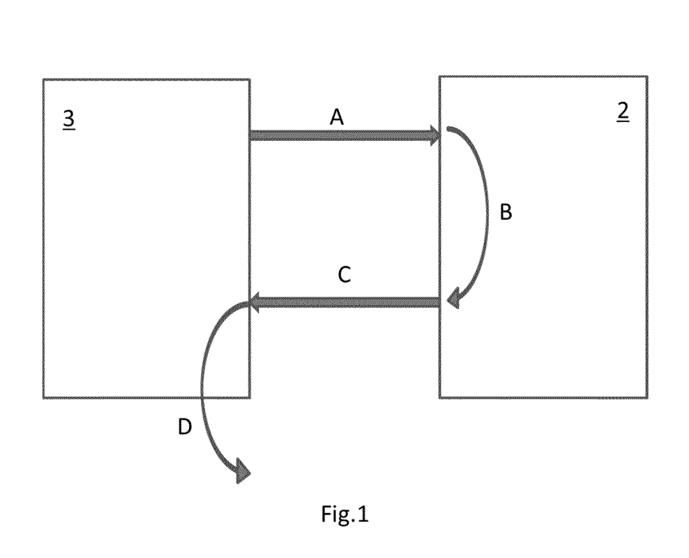

FIG. 1 schematically illustrates the method of the invention;

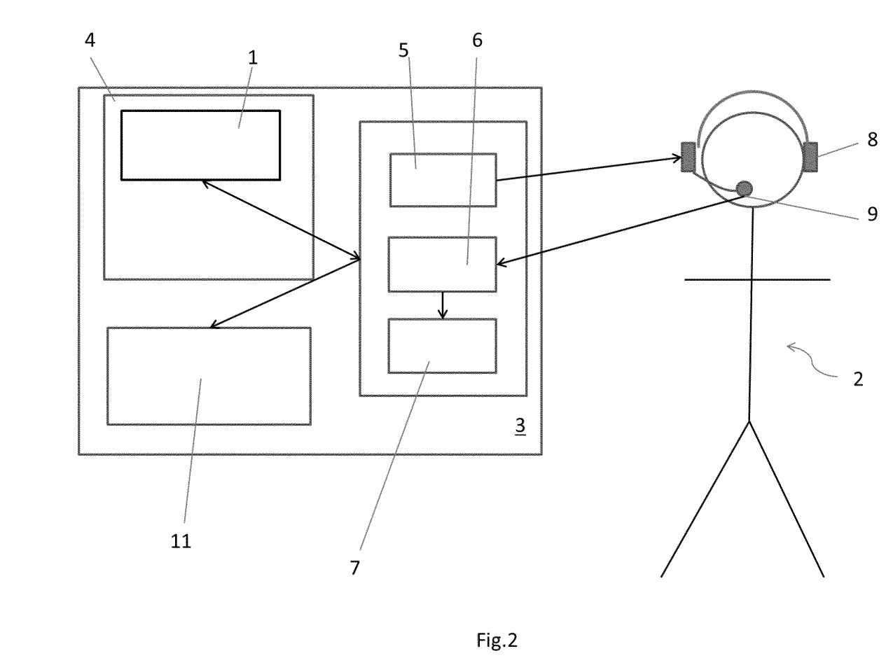

FIG. 2 illustrates the interface between the operator and the algorithm of the method in FIG. 1 and

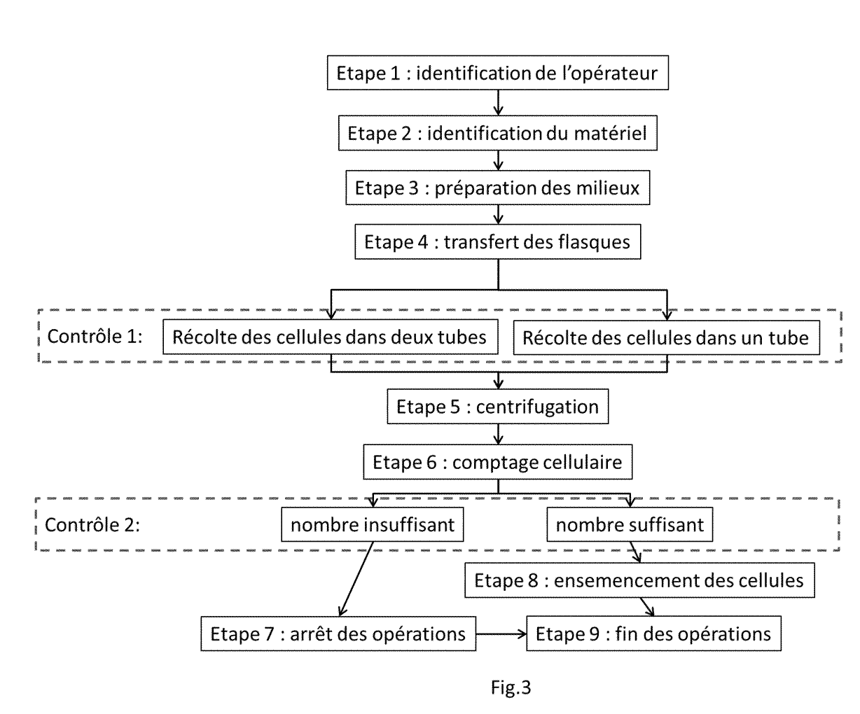

FIG. 3 illustrates the details of some of the steps of the method in FIG. 1.

With reference to FIG. 1, according to the task management method of a given recipe to be carried out by an operator 2 and comprising at least one task 1, an operator 2 interacts with a computer system 3 according to the following steps:
A: the execution order of the task is communicated verbally to the operator 2 by the computer system 3,
B: the operator performs the task, the operator verbally reports to the computer system on the execution of the task and
D: the execution of the task is verified by the computer system 3.

With reference to FIG. 2, the algorithm 10 for task management, allowing the implementation of the method of the invention, is installed on the computer system 3, which comprises a data storage unit 11 and wherein is specified the task 1 of the recipe 4 to be executed. The algorithm 10 comprises a speech synthesis module 5, a speech recognition module 6 and a control module 7. The operator 2 is equipped with a headset 8 and a microphone 9 interfacing with the computer system 3.

In concrete terms, the recipe 4, which has been entered into the computer system 3, comprises a number of tasks to be performed manually by the operator 2, with or without the use of tools. Each task 1 has a number of characteristics that must be monitored, measured, observed or applied by the operator.

In step A, using the speech synthesis module 5, the task 1, initially stored as digital data, is transcribed into a verbal execution order, which is transmitted by the audio system, in this case, a headset 8, to the operator 2. The headset 8 may advantageously communicate wirelessly with the computer system 3.

In step B, the operator 2 will perform the task 1. In the event that particular characteristics must be recorded by the operator 2, he may be asked, by another audio message, to provide information on these characteristics verbally, and this may be considered as a task in its own right.

Thus, in step C, the operator 2 will report the execution of the task 1 by stating a message into his microphone 9. His verbal message will be analyzed by the speech recognition module 6 and transcribed into digital data.

Here again, the speech recognition module is improved and comprises a specific grammar adapted to the recipe; for each message stated by the operator, a specific register or a series of specific response values according to the recipe is available to the computer system for the interpretation of the message. For example, if the operator has to enter a number, the speech recognition module 6 will only be able to transcribe the operator's message into a number. This feature is particularly useful when the operator works in a noisy environment and allows a much more reliable communication between the operator and the computer system. In order to further improve reliability, the computer system 3 invites the operator, by another verbal message, to confirm its correct interpretation of the message.

The message from the operator 2, relating to the execution of the task, may be of a varied nature. The operator may simply enter the status of the task, for example, that it is finished, give the numerical value of a measurement he has performed, or enter an observation he has made. It may also be provided that some operator messages have a particular function. For example, the operator could ask the computer system to repeat the task, or to take a break.

In step D, the control module 7 of the algorithm analyzes the content of the operator's 2 verbal message or report and decides on how to proceed. The analysis is carried out in particular by comparing this verbal report with the expected information previously provided in the recipe 4. The expected information may be a value, a range of values, a term or an expression. The computer system has an autonomous decision-making capacity resulting from the parameters of the recipe.

For example, if the operator 2 indicates that he has completed a task, the control module 7 may order the computer system 3 to communicate the next task. If the operator 2 has entered a numerical value, the control module 7 may perform a certain number of calculations incorporating this numerical value, compare it with its expected value and decide on the next instruction to be given to the operator, e. g. stopping operations if the deviation of the value from the expected value is too large or performing the next task if the value is in conformity. The criteria for conformity or non-conformity are defined when drafting the recipe 4.

Finally, the operator may also enter a characteristic that is non-critical to the production method, such as a simple observation, the expiration date of an ingredient or the reference of a piece of equipment. In all cases, the operator's 2 messages relating to task execution are recorded by the computer system 3 in a data storage unit 11 and may be used later, for example, to draft an execution report of a recipe or a quality report intended for regulatory authorities or a certificate of analysis for customers. A report on the tasks performed may be generated directly from the computer system 3.

The speech synthesis module 5 refers to a computer sound synthesis technique that allows artificial speech to be created from any text. Such a module comprises linguistic processing elements, in particular to transform the orthographic text into a pronounceable phonetic version without ambiguity, and signal processing elements to transform this phonetic version into digital sounds that may be listened to on a loudspeaker, for example here integrated into a headset 8. The speech recognition module 6 contains the inverse elements to transform a sound recorded by a microphone into digital text. Speech synthesis and speech recognition are technologies used to build speech interfaces and are used in particular for the vocalization of computer screens for blind people or on telephone speech servers.

It is also possible to couple these modules with a translation module, thus allowing the operator to choose the language in which he wishes to communicate with the computer system 3. This may be of interest when several production sites in different countries are to use the same method.

A simplified example of the method of the invention, applied to carrying out a cell seeding procedure, is shown in FIG. 3.

A recipe 4 for cell seeding, to be carried out in a sterile clean room, and containing several steps, was entered in the computer system 3. An operator 2, equipped according to the standards of the room where the tasks will be performed, has gathered the necessary equipment to carry out the recipe, possibly following a first recipe, not described here. In addition, he has equipped himself with an audio headset 8 and a microphone 9. When ready, he starts to execute the recipe, for example by saying “ready”.

The following dialog takes place between the computer system (SI) and the operator (verbal messages in italics):
SI: “Step 1, what is your name”
Operator: “Mr. X” (The name of the operator may be used to verify if he has all the valid authorizations necessary to perform this task, in real time, if the operator authorization database cooperates with the computer system allowing the method to be implemented, or later, if not.)
SI: “Step 2, enter the laminar flow code used for the operations”
Operator: “1256” (The equipment number may thus be associated with its maintenance profile, in real time, if the equipment maintenance database cooperates with the computer system allowing the method to be carried out, or later, if not.)
SI: “Enter the code of the temperature sensor used for the operations”
Operator: “1724”
SI: “Step 3: Prepare a 100-milliliter vial of human serum. Using the probe, measure the temperature of the vial and enter the temperature value”.
Operator: “37”
SI: “Step 4, transfer the flasks T175 under the laminar flow. Check 1: How many flasks did you transfer?”
Operator: “2”
Here, the number of flasks may affect the rest of the instructions, for example the adjustment parameters of the centrifuge.
SI: “Using a 50-milliliter pipette, transfer the cell suspension from each flask T175 into a 500-milliliter tube. Say “finished” when you have finished”
Operator: “Finished”
SI: “Step 5: Set the centrifuge according to the following parameters: acceleration 1600 g, temperature 20° C., duration 10 min. Put the tubes containing the cells in the centrifuge cup and start the centrifugation. Say “finished” when the centrifugation has finished.”
Operator: “Finished”
SI: “Step 6: With a 200-microliter micropipette, transfer 30 microliters of suspension into a 1.5-milliliter tube containing cells and 30 microliters of suspension into an empty tube. Add 30 microliters of PBS and 60 microliters of “Trypan blue” to each of the two tubes. Mix well and introduce 20 microliters of each dilution into a chamber of the Neubauer's chamber. Start the measurement. How many cells have been counted?”
Operator: “1732”

The control module will correct the value of 1732 with the dilution coefficients used, and possibly the parameters related to the particular equipment used, and then compare the result obtained with the expected values for this measurement. For example, if the calculated value is lower than the values of the expected range, and in this case the process must be stopped, the following message may be issued:
SI: “Check 2: The number of cells is insufficient. Step 7: Dispose of the cells in the bio-waste bin. Say “finished” when you have finished”
Operator: “Finished”

If the number of cells had been sufficient, the seeding instructions in Step 8 could have been given.
SI: “Step 9: This is the end of the operations in the laminar flow, please clean up your work surface. Carry out the environmental sampling corresponding to the end of operations. Say finished when you have finished and then leave the room.”
Operator: “Finished”.

Concurrently with this dialogue between the operator 2 and the computer system 3, all the information received from the operator has been recorded in the data storage unit 11. This information may be used by those in charge of reporting operations, validating a batch, or tracking all the elements that have intervened in the production of a product, for example by those in the quality department or those in charge of production who want to understand why the cell seeding has failed. This information may also be used to generate statistics.

It is not excluded that in some cases, the recipe may require the use of an external controller, such as the production manager or team leader. In this case, a function may be provided on the computer system so that the operator in the sterile room may communicate verbally with the external controller positioned outside.

Advantageously, the computer system 3 may also comprise a module to help in drafting the recipe 4. This module may, for example, propose predefined steps wherein some parameters may be modified. This configuration, which limits to a certain extent the range of grammar that may be used by the drafter of the recipe, helps in particular to limit errors in the interpretation of tasks. It may also enable the drafter of the recipe to remain in compliance with the standards or rules in force by preventing him from leaving the regulatory framework and by predefining a certain number of verification steps.

It is apparent that the method described above is not limited to biological operations in a sterile room but may be applied to many environments. An example is the nuclear sector, where strict procedures must be implemented to limit operators' exposure to radioactive radiation. The analysis of the quantities of radiation received by the operator could, for example, determine the number of tasks to be accomplished, while taking into account the time required to carry out the essential tasks at the end of the procedure, i.e. decontamination.

The method of the invention saves time by allowing the computer system to make impartial decisions in real time on the steps of the recipe to be implemented based on the information provided by the operator, without having to enter information on a computer or write it on paper, which must then be decontaminated, whereas a simple headset 8 and a microphone 9 may easily be inserted into his equipment.

1. Method for managing tasks of a given recipe (4) to be carried out by at least one operator (2) in a regulatory environment, whereby
the operator receives from a computer system (3) voice commands (1) to execute the tasks of the recipe (4),
the operator (2) executes the tasks (1),
the operator (2) reports verbally to the computer system on the task (1) execution and
the task (1) execution is verified through the computer system (3), the verification of the task (1) execution comprising comparing the information reported by the operator (2) with the expected information previously entered in the recipe (4), said expected information being one of the elements of the group comprising a value, a range of values, a term and an expression, and the verification integrating the result of the task (1) verification in the selection of a new order to be issued.

2. Method according to claim 1 whereby a dialogue is established between the operator (2) and the computer system (3).

3. A method according to claim 1, whereby the recipe (4) is previously entered in the computer system (3).

4. Method according to claim 1, whereby the computer system (3) cooperates with other computer systems for the verification of the task (1) execution.

5. Method according to claim 1, whereby the computer system (3) generates a report relating to the task (1) execution.

6. Method according to claim 1, whereby the task (1) execution is stored via the computer system (3).

7. Algorithm (10) for managing the tasks (1) of a given recipe (4) to be carried out by at least one operator (2) in a regulatory environment for the implementation of the method of claim 1, designed to
issue voice commands to the operator (2) to execute the tasks (1) of the recipe (4),
receive a verbal report on the task (1) execution and
participate in the verification of the task (1) execution.

8. Algorithm (10) according to claim 7, designed to integrate the result of the verification of the tasks (1) into the selection of a new order to be issued.

9. Algorithm (10) according to claim 7, comprising a module (4) to aid in drafting the recipe.

10. Algorithm (10) according to claim 7, comprising a speech recognition module (6) associated with a specific grammar to receive the verbal report.

11. Algorithm (10) according to claim 7, designed to integrate external data when verifying the task (1) execution.

12. Algorithm (10) according to claim 7, wherein the algorithm (10) analyzes the content of the operator's (2) verbal report by comparison with expected information, previously entered in the recipe (4), said expected information being one of the elements of the group comprising a value, a range of values, a term and an expression.

13. Algorithm (10) according to claim 7, designed to aid in the drafting of a report relating to the task execution.
